(19) United States
(12) Patent Application Publication
(10) Pub. No.: US 2011/0070648 A1
(43) Pub. Date: Mar. 24, 2011

(54) METHOD FOR CELL EXPANSION

(75) Inventors: Cecilia Anneren, Uppsala (SE); Linnea Pauler, Uppsala (SE)

(73) Assignee: GE HEALTHCARE BIO-SCIENCES AB, UPPSALA (SE)

(21) Appl. No.: 12/991,930

(22) PCT Filed: May 11, 2009
(86) PCT No.: PCT/SE2009/050517

§ 371 Date: Nov. 10, 2010

(30) Foreign Application Priority Data
May 15, 2008 (SE) 0801117-3

(51) Int. Cl. C12N 5/071; C12N 5/0775; C12N 5/078

(52) U.S. Cl. 435/380; 435/378

(57) ABSTRACT

The present invention relates to a method for cell expansion. More closely, it relates to a method for expansion of cells, such as mesenchymal stem cells, on microcarriers in a plastic bag bioreactor. The invention enables expansion to therapeutic amounts of stem cells. The method comprises the following steps: a) addition of cells in cell culture medium and microcarriers to a plastic bag container; b) allowing the cells to adhere to the microcarriers while the container is kept substantially still; c) addition of further cell culture medium once the cells have adhered; d) culturing the cells under gentle and constant agitation; e) increase the surface area for continued culturing; and f) final harvesting of cells by an active detachment and separation step.

### CROSS-REFERENCE TO RELATED APPLICATIONS

This application is a filing under 35 U.S.C. §371 and claims priority to international patent application number PCT/SE2009/050517 filed May 11, 2009, published on Nov. 19, 2009 as WO 2009/139703, which claims priority to application number 0801117-3 filed in Sweden on May 15, 2008.

### FIELD OF THE INVENTION

The present invention relates to a method for cell expansion. More closely, it relates to a method for cell expansion of sensitive cells, such as mesenchymal stem cells, on microcarriers in a plastic bag bioreactor.

### BACKGROUND OF THE INVENTION

Cell culture and expansion is a very important step in cell therapy and mesenchymal stem cells (MSCs) represent the second most selected stem cell group (after haematopoietic stem cells) in the adult stem cell market today.

A recent upsurge in the use of primary and stem cells (e.g. MSCs) for cell based therapy has created a growing need for cell culture systems that can be used to expand these cell types. Typically, MSCs are grown in monolayer T-flask cultures, which is labour intensive and space requiring if large amount of cells are to be produced. There is one article describing successful culture of porcine bone-marrow derived MSCs on microcarriers in spinner flasks (Frauenschuh, S., E. Reichmann, et al. (2007). “A Microcarrier-Based Cultivation System for Expansion of Primary Mesenchymal Stem Cells.” Biotechnol Prog 23(1): 187-193.) However, this is not a disposable system and the shear forces from the mechanical mixing may damage the cells.

Previous published results have shown that it is possible to expand various types of cells (e.g. Vero cells and MDCK cells) on microcarriers in the Wave Bioreactor (Genzel, Y., R. M. Olmer, et al. (2006). “Wave microcarrier cultivation of MDCK cells for influenza virus production in serum containing and serum-free media.” Vaccine 24(35-36): 6074-87.). However, the applications for these cultures lie in the vaccine production area. Similar protocols cannot be applied to primary cell cultivation since they differ from cancer cell lines in several ways.

For instance, MSCs are more sensitive and proliferative properties are limited as they loose their multilineage capacity after certain number of passages.

US 2007-0264713 relates to a method for proliferating stem cells on microcarriers. The stem cells, the microcarriers as well as culture medium are introduced into a container which could be a spinner flask or a bioreactor. An intermittent agitation technique is used in the method, wherein the medium is moved between 10 and 100 minutes and not moved between 10 and 60 minutes.

The method is said to provide large yields of stem cells.

However, there is still a need of a new method suitable for expanding adherent cells, such as primary cells and stem cells, in bioreactors, for clinical scale production.

### SUMMARY OF THE INVENTION

The present invention relates to cell expansion by a method capable of starting from a small volume with a low number of cells and ending with high numbers of cells suitable for, for example, cell therapy.

The invention relates to a method for cell expansion, comprising the following steps: a) addition of cells in cell culture medium and microcarriers to a plastic bag container; b) allowing the cells to adhere to the microcarriers while the plastic bag container is kept substantially still; c) addition of further cell culture medium once the cells have adhered; d) culturing the cells under gentle (<7 rpm) and constant agitation, preferably to 70-80% confluency; e) increase the surface area for continued culturing through either an active detachment step of cells from the microcarriers followed by addition of new carriers, or by adding new carriers and allowing spontaneous migration of cells to these and f) final harvesting of cells by an active detachment and separation step.

The active detachment step in e) may, for example, be by allowing the cells to detach by using enzymes, thermo responsive agents and/or pH responsive agents. Alternatively, the cells are expanded by allowing passive migration from confluent beads to newly added beads.

Step b) may be performed with intermittent or occasional rocking under very low speed, more gentle than in step c).

Preferably, the step e) is repeated at least once.

In a preferred embodiment, the core of microcarriers are provided with magnetic particles to facilitate sedimentation and/or decantation of culture media etc.

It is critical that the volume in step a) is low to increase cell-to-microcarrier contact and preferably, the volume of the added cells and microcarriers in step a) is between 150-300 ml. Seeding density is 1-10 cells per microcarrier and the start amount of microcarrier should be at least 0.2 g (dry weight) per litre.

The cells are primary cells or stem cells. The stem cells may be adult or embryonic or induced pluripotent stem cells, iPS. The cells may be nucleated cells from adipose tissue, bone marrow or cord blood.

In one embodiment, the cells are pre-cultivated before step a). This is preferably done in a separate container, such as a culture flask.

In a preferred embodiment, the cells are adult mesenchymal stem cells. In a preferred embodiment, a final 3-5 g (dry weight) microcarriers per litre are present, which leads to a final cell number of 300-500×10⁶ MSCs in a 1 litre bioreactor.

Preferably steps e) and f) are repeated until 3-8 g microcarriers per litre medium is reached. Up to 5 g without perfusion and up to 8 g with perfusion of the bioreactor bag with fresh medium etc.

In another embodiment, the MSCs may be obtained from a purified blood (mononuclear cell fraction) or tissue sample, without any pre-cultivation. Thus, these cells may be provided directly into the above workflow.

Preferably, the container is an inflated bioreactor bag. The cultivation may be performed under hypoxic conditions.

The cells may be detached in step e) inside the container or outside the container. In the latter case the cells and microcarriers are re-introduced into the container.

The final harvest of the expanded cells is preferably performed by the same principle as detachment outside the bag which is described more closely in the detailed section below.

### BRIEF DESCRIPTION OF THE DRAWINGS

FIG. 1 is a schematic view of a cell expansion bag put in an upright position, allowing the microcarriers to sediment down to one corner of the bag. The carriers are transferred to an external device for washing and trypsination (2A). Alternatively, media is pumped out from the bag while carriers remain in the bag by including a filter in the transfer tube (2B). Washing and trypsination are then performed inside the bag.

FIG. 2 shows the growth of bone marrow-derived MSCs on microcarriers in a plastic bag bioreactor after 1 day (A) and after 6 days (B), respectively. Cells migrate to empty carriers. Fresh empty carriers were added to an almost confluent MSC culture increasing the amount of carriers with 50% (e.g. 25% of all carriers were empty at time 0). After 24 hours 16% of the carriers were empty (black bars) and at 4 days only 1% empty carriers were found in the culture.

### DETAILED DESCRIPTION OF THE INVENTION

The invention will now be described more fully in association with the accompanying drawings. However, it is to be understood that the examples only are intended for illustrative purposes without limiting the scope of the invention.

In the experiments a bioreactor has been used which provides a closed disposable system. Cells are grown on microcarriers which are agitated by a gentle wave-like motion and the surface area is increased by adding new carriers to the cultures as the cells expand. The surface area per gram of the preferred microcarriers CYTODEX™ 1 and CYTODEX™ 3 is 4400 cm² for CYTODEX™ 1 and 2700 cm² for CYTODEX™ 3.

One therapeutic cell dose can be produced using one single 2 L bag. The method according to the invention is suitable for the production of therapeutic stem cells.

### Examples

A purified patient tissue sample of cells (approximately 1×10⁶ MSCs) is pre-cultured in a T-flask to achieve an amount of 5-10×10⁶ MSCs. The MSCs and 0.2-1 g dry weight (1-10 cells/bead) pre-swollen, sterilized CYTODEX™ (GE Healthcare Bio-Sciences AB), equilibrated in a commercially available MSC media, is added to an inflated 2 L Wave bag (GE Healthcare Bio-Sciences).

The cells are allowed to adhere to the microcarriers either in the incubator during static conditions. Initially, media volume is kept low in the bag (150-300 ml) to increase cell-to-bead contact. Once the cells have attached, more media is added to achieve desired volume. During the culturing, a constant low rocking speed (<7 rpm) and rocking angle (≦5°) is used.

When the cells have reached 70-80% confluency (approximately one week culture period, see FIG. 2B), they are detached by e.g. a trypsin-based method. The trypsination can either be performed in the bag or the microcarriers can be transferred to an external bottle/smaller bag for trypsination.

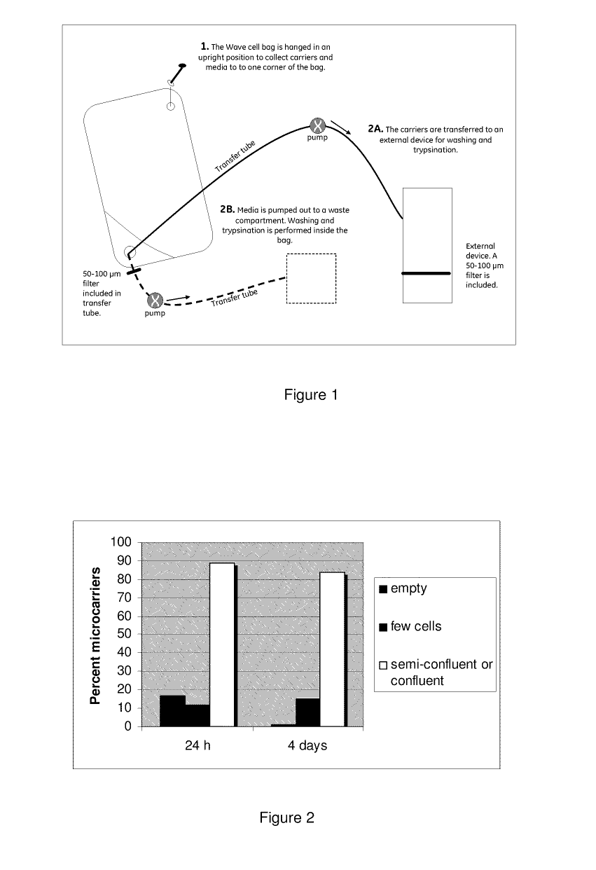

Outside the bag: The Wave bag is put in an upright position, allowing the microcarriers to sediment down to one corner of the bag (FIG. 1, 2A). The carriers are transferred by gravity flow or pump to an external device, which includes a 50-100 μm filter for washing and trypsination steps. Advantage: easier to wash the carriers in an external device.
Inside the bag: The Wave bag is put in an upright position and carriers are allowed to sediment down to one corner of the bag (FIG. 1, 2B). Media is removed and the washing and trypsination is performed inside the bag. Advantage: No removal of carriers and cells from the Wave bag, thus, using one single compartment during the entire culture. To avoid complicated and time-consuming washing and to prevent loss of microcarriers, a 50-100 μm filter is inserted between the transfer tube of the bag and the waste outlet for media removal.

In both the above situations (outside and inside the bag, respectively) and in other cell cultivation situations where sedimentation is desired, the sedimentation of the microcarriers may be enhanced by adding magnetic particles, such as Fe₂O₃, to make the microcarriers more heavy, which also facilitates decanting of cell culture media etc. from the plastic bag. Even more efficient sedimentation and/or decanting is achieved if an external magnet is used to immobilize the microcarriers during the decanting procedure.

When trypsination is completed, new carriers are added to increase surface area and cells are allowed to attach once again for further proliferation. The detachment and subsequently the adding of new carriers and media is repeated until 3 gram CYTODEX™ carriers in one litre media is reached with a split ratio of approximately 1:3. The final harvest of the cells is performed by the same principle as trypsination outside the bag.

Trypsination and addition of new carriers to increase surface area can be exchanged by adding new carriers directly to the culture and allowing the cells to migrate over to new carriers. Four days after addition of empty microcarriers to a MSC culture most carriers in the culture are populated with cells (FIG. 2). The method of adding new carriers to a culture without a prior detachment step is particularly useful for MSCs, which are highly migratory in nature which was demonstrated as follows. GFP expressing MSCs and wilt-type MSCs were seeded on CYTODEX™ 1 carriers in separate compartments, allowed to attached and then subsequently pooled after 24 hours. After a three-day culture the cells were completely intermingled and both GFP expressing and wild-type cells were found on most carriers (results not shown).

It is apparent that many modifications and variations of the invention as hereinabove set forth may be made without departing from the spirit and scope thereof. The specific embodiments described are given by way of example only, and the invention is limited only by the terms of the appended claims.

1. A method for cell expansion, comprising the following steps:
a) addition of cells in cell culture medium and microcarriers to a plastic bag container;
b) allowing the cells to adhere to the microcarriers while the plastic bag container is kept substantially still;
c) addition of further cell culture medium once the cells have adhered;
d) culturing the cells under gentle (<7 rpm) and constant agitation;
e) increase the surface area for continued culturing; and
f) final harvesting of cells by an active detachment and separation step.

2. The method of claim 1, wherein step e) is repeated at least once.

3. The method of claim 1, comprising in step e) adding new carriers and allowing spontaneous migration of cells to the new carriers.

4. The method of claim 1, comprising in step e) an active detachment step of cells from the microcarriers and addition of new carriers.

5. The method of claim 1, wherein the microcarriers are provided with magnetic particles.

6. The method of claim 1, wherein the volume of the added cells and microcarriers in step a) is between 150-300 ml, the seeding density is 1-10 cells /microcarrier and the amount of microcarriers is at least 0.2 g (dry weight) per litre.

7. The method of claim 1, wherein the cells are nucleated cells from adipose tissue, bone marrow or cord blood.

8. The method of claim 7, wherein the cells are adult mesenchymal stem cells.

9. The method of claim 1, wherein steps e) and f) are repeated until 3-8 g microcarriers per litre medium is reached.

10. The method of claim 1, wherein the container is an inflated bioreactor bag.

11. The method of claim 1, wherein the cells are allowed to detach in step e) inside the container.

12. The method of claim 1, wherein the cells are allowed to detach in step e) outside the container, after which the cells and microcarriers are re-introduced into the container.

13. The method of claim 1, wherein the cells are allowed to detach by using enzymes, thermo responsive agents and/or pH responsive agents.

14. The method of claim 1, wherein the cells are expanded by allowing passive migration from confluent beads to newly added beads.
